(19) United States
(12) Patent Application Publication
(10) Pub. No.: US 2024/0197672 A1
(43) Pub. Date: Jun. 20, 2024

(54) COMPOSITION FOR INHIBITING SKIN PIGMENTATION THROUGH AUTOPHAGY ACTIVITY CONTAINING PROCYANIDIN A2 AND QUERCETIN, AS ACTIVE INGREDIENTS

(71) Applicant: KYUNGPOOK NATIONAL UNIVERSITY INDUSTRY-ACADEMIC COOPERATION FOUNDATION, Daegu (KR)

(72) Inventors: Sang-Han LEE, Daegu (KR); Borim SONG, Daegu (KR)

(21) Appl. No.: 18/515,486

(22) Filed: Nov. 21, 2023

(30) Foreign Application Priority Data
Nov. 21, 2022 (KR) 10-2022-0156116

(51) Int. Cl. A61K 31/353; A61K 8/49; A61P 17/00; A61Q 19/02

(52) U.S. Cl. CPC A61K 31/353; A61K 8/498; A61P 17/00; A61Q 19/02

(57) ABSTRACT

The present invention relates to a composition for inhibiting skin pigmentation through enhanced autophagy activity, comprising procyanidin A2 and quercetin as active ingredients. The combination of procyanidin A2 and quercetin compounds not only inhibits tyrosinase but also increases autophagy activity, thus showing remarkable effectiveness in inhibiting melanin synthesis. Therefore, this composition can be advantageously used in various materials, including food products, cosmetic compositions, and pharmaceutical compositions, associated with skin pigmentation.

### TECHNICAL FIELD

The present invention relates to a composition for inhibiting skin pigmentation through enhanced autophagy activity, comprising procyanidin A2 and quercetin as active ingredients.

### BACKGROUND ART

Melanin, produced by melanocytes, is a dark brown pigment found in the skin and eyes of animals and determines human skin and hair color. While protecting the body from ultraviolet rays, excessive accumulation of melanin in the skin can lead to pigmentation disorders like freckles and melasma, as well as skin aging and cancer. Due to the fact that it can lead to skin cancer and people's increasing desire for aesthetics, inhibiting melanin production has become a major target in the fragrance industry.

Melanin is a polymer of phenolic compounds widely found in nature, a complex of black pigment and proteins. The general process of melanogenesis starts in the melanosomes, organelles in melanocytes, with the amino acid tyrosine, which becomes DOPA, then oxidizes to DOPA-quinone, involving the oxidase enzyme tyrosinase. The produced melanin is transferred in vesicle form, called melanosomes, by the dendrites of melanocytes to the surrounding keratinocytes, spreading evenly across the epidermis.

Autophagy refers to the phenomenon where materials within a cell are removed by the cell itself, and is also termed self-eating. This self-eating process functions to eliminate cellular waste, degenerative proteins, or organelles that have reached the end of their lifespan or have become dysfunctional. Proteins/organelles targeted for removal are isolated within a cell in a vesicle called an autophagosome, which is enclosed by a double membrane. This vesicle then combines with a lysosome to form an autophagolysosome, where the lysosomal enzymes decompose the contents.

Procyanidin A2 and quercetin are flavonoids abundantly present in nature. Flavonoids are known to act as free radical scavengers, antioxidants, superoxide anion absorbers, ultraviolet light absorbers, and lipid peroxyl radical scavengers. They have also been shown to reinforce collagen structure, exhibit antimutagenic, anti-inflammatory, and antiviral effects.

However, no research has been reported on the specific types of flavonoids that show excellent effects in inhibiting pigmentation through tyrosinase inhibition and autophagy activation mechanisms, nor on their combinations and optimized conditions.

### PRIOR ART DOCUMENT

### Patent Literature

(Patent Document 1) KR No. 10-2021-0021828

### DISCLOSURE

### Technical Problem

The present invention is directed to providing a health functional food composition for skin whitening or preventing or alleviating skin pigmentation, comprising procyanidin A2 and quercetin as active ingredients.

The present invention is also directed to providing a functional food composition for skin whitening or preventing or alleviating skin pigmentation, comprising procyanidin A2 and quercetin as active ingredients.

Furthermore, the present invention is directed to providing a cosmetic composition for skin whitening or preventing or alleviating skin pigmentation, comprising procyanidin A2 and quercetin as active ingredients.

Additionally, the present invention is directed to providing a quasi-drug composition for skin whitening or preventing or alleviating skin pigmentation, comprising procyanidin A2 and quercetin as active ingredients.

Moreover, the present invention is directed to providing a pharmaceutical composition for preventing or treating skin pigmentation disorders, comprising procyanidin A2 and quercetin as active ingredients.

However, the technical problem to be solved by the present invention is not limited to the aforementioned issues. Other problems not mentioned here will be clearly understood by those skilled in the art from the description below.

### Technical Solution

The present invention provides a health functional food composition for skin whitening or preventing or alleviating skin pigmentation, comprising procyanidin A2 and quercetin as active ingredients.

The present invention also provides a functional food composition for skin whitening or preventing or alleviating skin pigmentation, comprising procyanidin A2 and quercetin as active ingredients.

In an embodiment of the present invention, the procyanidin A2 and quercetin may be in a weight ratio of 1:2 to 2:1, but are not limited thereto.

In an embodiment of the present invention, the composition may comprise procyanidin A2 and quercetin each at a concentration of 20 μM, but are not limited thereto.

In an embodiment of the present invention, the procyanidin A2 may be in an amount greater than 0 and less than or equal to 50 μM, but is not limited thereto.

In an embodiment of the present invention, the quercetin may be in an amount greater than 0 and less than or equal to 25 μM, but is not limited thereto.

In an embodiment of the present invention, the composition may inhibit the activity of tyrosinase, but is not limited thereto.

In an embodiment of the present invention, the composition may inhibit the amount of melanin produced, but is not limited thereto.

In an embodiment of the present invention, the composition may decrease the expression of one or more melanin synthesis-related genes selected from the group consisting of tyrosinase, TRP-1, TRP-2, and MITF, but is not limited thereto.

In an embodiment of the present invention, the composition may increase the expression of at least one gene of (a), and decreases the expression of at least one gene of (b), but is not limited thereto:
(a) LC3-II, ATG5, and Beclin-1; and
(b) p62, and LC3-I.

In an embodiment of the present invention, the composition may inhibit an autophagy inhibitor, but is not limited thereto.

In an embodiment of the present invention, the autophagy inhibitor may be one or more selected from the group consisting of wortmannin, bafilomycin, and 3-Methyladenine (3-MA), but is not limited thereto.

The present invention provides a cosmetic composition for skin whitening or preventing or alleviating skin pigmentation, comprising procyanidin A2 and quercetin as active ingredients.

In an embodiment of the present invention, the cosmetic may be at least one selected from the group consisting of flexible cosmetic water, nutrition cosmetic water, gel, essence, spray essence, emulsion, cream, lotion, powder, soap, surfactant-containing cleansing, oil, powder foundation, emulsion foundation, wax foundation, cleanser and bath powder, mask pack, massage cream, hand cream, sunscreen, body lotion, and body cleanser, but is not limited thereto.

The present invention provides a quasi-drug composition for skin whitening or preventing or alleviating skin pigmentation, comprising procyanidin A2 and quercetin as active ingredients.

In an embodiment of the present invention, the quasi-drug composition may be for non-oral administration, but is not limited thereto.

The present invention provides a pharmaceutical composition for preventing or treating skin pigmentation disorders, comprising procyanidin A2 and quercetin as active ingredients.

In an embodiment of the present invention, the skin pigmentation disorder may be one or more selected from the group consisting of melasma, freckles, lentigo, seborrheic keratosis, mole, café-au-lait spots, nevus of Ota, blue nevus, hyperpigmented spots, drug-induced hyperpigmentation, gravidic chloasma, and post-inflammatory hyperpigmentation due to wounds or dermatitis, but is not limited thereto.

Furthermore, the present invention provides a method for skin whitening, or preventing or alleviating skin pigmentation disorders, which comprises the step of a subject in need thereof consuming a composition comprising procyanidin A2 and quercetin as active ingredients.

Additionally, the present invention provides the use of a composition comprising procyanidin A2 and quercetin as active ingredients for manufacturing health functional foods or functional foods for skin whitening, or preventing or alleviating skin pigmentation disorders.

Moreover, the present invention provides a method for skin whitening, which comprises the step of treating the skin of a subject in need thereof with a composition comprising procyanidin A2 and quercetin as active ingredients.

Furthermore, the present invention provides the use of a composition comprising procyanidin A2 and quercetin as active ingredients for skin whitening.

Additionally, the present invention provides the use of a composition comprising procyanidin A2 and quercetin as active ingredients for manufacturing cosmetic preparations for skin whitening.

Furthermore, the present invention provides a method for preventing, alleviating, or treating skin pigmentation disorders, which comprises the step of administering a composition comprising procyanidin A2 and quercetin as active ingredients to a subject in need thereof.

Additionally, the present invention provides the use of a composition comprising procyanidin A2 and quercetin as active ingredients for preventing, alleviating, or treating skin pigmentation disorders.

Moreover, the present invention provides the use of a composition comprising procyanidin A2 and quercetin as active ingredients for manufacturing preparations for preventing, alleviating, or treating skin pigmentation disorders.

### Advantageous Effects

According to the composition for inhibiting skin pigmentation exhibiting autophagy enhancing activity, comprising procyanidin A2 and quercetin as active ingredients, the combination of procyanidin A2 and quercetin compounds not only inhibits tyrosinase but also increases autophagy activity, thereby exhibiting excellent efficacy in inhibiting melanin synthesis and thus preventing skin pigmentation. This makes it useful in various materials, including food products, cosmetic compositions, and pharmaceutical compositions, related to skin pigmentation.

### BRIEF DESCRIPTION OF THE DRAWINGS

FIG. 1 is a graph showing the results of the tyrosinase inhibition activity experiment for the control group, arbutin, procyanidin A2, and quercetin respectively.

FIG. 2 is a graph showing the results of the tyrosinase inhibition activity analysis for the control group, procyanidin A2, quercetin, and the combination of both compounds.

FIG. 3 is a graph showing the cytotoxicity results according to the concentrations of the control group, procyanidin A2, quercetin, and the combination of both compounds.

FIGS. 4A and 4B are graphs and photographs showing the results of the melanin synthesis inhibition activity analysis for the negative control group, positive control group, procyanidin A2, quercetin, and the combination of both compounds.

FIG. 5 is a graph showing the results of the analysis of the inhibitory activity on the expression of melanin synthesis-related genes tyrosinase, TRP-1, TRP-2, and MITF following treatment with the control group, arbutin, procyanidin A2, quercetin, and the combination of both compounds.

FIG. 6 is a graph showing the results of the analysis of the increase or decrease in expression of autophagy-related genes LC3-II, ATG5, Beclin-1, p62, and LC3-I following treatment with the control group, arbutin, procyanidin A2, quercetin, and the combination of both compounds.

FIG. 7 is a graph showing the results of analyzing the melanin synthesis inhibition effect of the combination of both compounds and treatment with an autophagy inhibitor (wortmannin, bafilomycin, and 3-MA) according to an embodiment of the present invention. And this is a graph showing the results of analyzing the effect of inhibiting melanin synthesis when simultaneously treating the combination and autophagy inhibitors.

### MODES OF THE INVENTION

The present invention provides a health functional food composition for skin whitening or preventing or alleviating skin pigmentation, comprising procyanidin A2 and quercetin as active ingredients.

The present invention provides a functional food composition for skin whitening or preventing or alleviating skin pigmentation, comprising procyanidin A2 and quercetin as active ingredients.

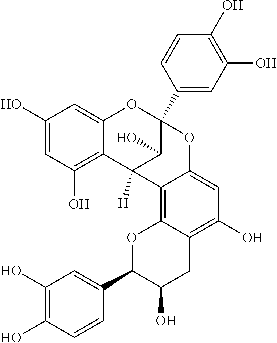 [SMILES: Oc1cc(O)c2c(c1)O[C@@]1(c3ccc(O)c(O)c3)Oc3cc(O)c4c(c3[C@@H]2[C@H]1O)O[C@H](c1ccc(O)c(O)c1)[C@H](O)C4]

In the present invention, procyanidin A2 may be represented by the above structural formula but is not limited thereto and may be represented by the broadest range of structural formulas or chemical formulas known in the art.

Additionally, in the present invention, procyanidin A2 may include all food acceptable, pharmaceutically acceptable, and cosmetically acceptable salts of procyanidin A2, but is not limited thereto.

Furthermore, procyanidin A2 in the present invention may include a variety of compounds among intermediates, secondary products, etc., appearing through metabolism or chemical reactions inside or outside the human body, which substantially have the same structure, functional groups, and function as procyanidin A2.

Procyanidin is one of the wide-ranging proanthocyanidins present in plants. Proanthocyanidins are a group of compounds formed by the condensation or polymerization of flavan-3-ol or flavan-3,4-diol units and are a type of condensed tannin. Among these, procyanidin encompasses oligomers and polymers of dimers or higher, having catechin, epicatechin, catechin gallate, epicatechin gallate, gallocatechin gallate, or epigallocatechin gallate as their basic backbone.

Procyanidins are classified into various types depending on their basic backbone and bonding form. For example, procyanidin B3 has a structure of catechin-(4α→8)-catechin, and procyanidin B1 has a structure of epicatechin-(4β→8)-catechin. Procyanidin B2 has a structure of epicatechin-(4β→8)-epicatechin, and procyanidin B5 has a structure of epicatechin-(4β→6)-epicatechin (Walter Feucht et al., Z. Naturforsch., 54c, 942-945, 1999).

Procyanidins may be obtained by solvent extraction from various types of plants or by separation and purification using chromatography, and it is known that they may also be separated from wine or beer. There are reports of procyanidins being isolated by solvent extraction and chromatography from adzuki beans (Vigna angularis Ohwi et Ohashi) (Toshiaki Ariga et al., Agricultural Biological Chemistry, Vol. 45, 2709-2712, 1981), loblolly pine bark (R. W. Hemingway et al., Phytochemistry, Vol. 22, 275-281, 1983), and the roots of Polygonum multiflorum (Nonaka et al., Phytochemistry, Vol. 21, 429-432, 1982).

In the present invention, quercetin may be represented by the above structural formula but is not limited thereto and may be represented by the broadest range of structural formulas or chemical formulas known in the art.

Additionally, quercetin in the present invention includes all food acceptable, pharmaceutically acceptable, and cosmetically acceptable salts of quercetin, but is not limited thereto.

Furthermore, quercetin in the present invention may include a variety of compounds among intermediates, secondary products, etc., appearing through metabolism or chemical reactions inside or outside the human body, which substantially have the same structure, functional groups, and function as quercetin.

Quercetin is a compound well-known as a flavonoid component included in licorice extract, with its IUPAC name being 2-(3,4-dihydroxyphenyl)-3,5,7-trihydroxy-4H-chromen-4-one, and its molecular formula represented as C₁₅H₁₀O₇₂H₂O.

Quercetin is a type of flavonoid, one of the flavonoids having a 3-hydroxyflavone backbone structure (3-hydroxy-2-phenylchromen-4-one), and is known to be contained as an active ingredient in various types of medicinal plants, including licorice, apple peels, and onion skins.

Quercetin is reported to be able to damage oncoviral replication, specifically inhibiting the secretion of HBsAg and HBeAg in cells infected with hepatitis virus B (HBV). Additionally, quercetin is effective in inhibiting HCV replication and may be used in combination with interferon a as an anti-HCV therapeutic. Furthermore, research is being conducted to use quercetin and its analogs as HIV-1 reverse transcriptase inhibitors in HIV treatments.

There are also reports that quercetin may be used as an anticancer therapeutic. This is attributed to quercetin's antioxidant or anti-inflammatory activity, and it also has the ability to inhibit the growth of cancer cells and induce apoptosis (cell death) (Dajas F. Life or death: neuroprotective and anticancer effects of quercetin. Journal of ethnopharmacology. 2012; 143:383-396).

In an embodiment of the present invention, the procyanidin A2 and quercetin may be in a weight ratio of 1:2 to 2:1, but are not limited thereto.

When the procyanidin A2 and quercetin are mixed in a weight ratio of 1:1 or more, for example, in an embodiment of the present invention, when mixed in a weight ratio of 1:2 or 2:1, since the tyrosinase inhibition activity is similar, a 1:1 weight ratio may be the most efficient, but is not limited thereto.

In an embodiment of the present invention, the composition may comprise procyanidin A2 and quercetin each at a concentration of 20 μM, but is not limited thereto.

In the present invention, the composition may have the highest tyrosinase inhibitory activity efficiency when procyanidin A2 and quercetin are mixed in a 1:1 weight ratio, which may refer to, but is not limited to, a 1:1 weight ratio composition wherein 20 μM of each compound is mixed. Therefore, the composition of the present invention may include all cases where the compounds of procyanidin A2 and quercetin are mixed in a 1:1 weight ratio exceeding 0 μM each.

In an embodiment of the present invention, the procyanidin A2 may be in an amount greater than 0 and less than or equal to 50 μM, but is not limited thereto.

In an embodiment of the present invention, the quercetin may be in an amount greater than 0 and less than or equal to 25 μM, but is not limited thereto.

According to an embodiment of the present invention, procyanidin A2 and quercetin may show cytotoxicity at concentrations exceeding 50 μM and 25 μM respectively, or at concentrations of 100 μM and 50 μM or more, but are not limited thereto.

In an embodiment of the present invention, the composition may inhibit the activity of tyrosinase, but is not limited thereto.

In the present invention, tyrosinase is an oxidase enzyme that regulates the production of melanin. This enzyme is primarily involved in two melanin synthesis reactions. The first is the hydroxylation of monophenol and the second is the conversion of o-diphenol to its corresponding o-quinone. o-Quinone may undergo several reactions for melanin formation.

Tyrosinase is a copper-containing enzyme and may appear in tissues of plants and animals. As seen in the browning of chopped or peeled potatoes, it can catalyze the production of melanin and tyrosine from precursors and may be found in melanosomes where melanin is synthesized in skin melanocytes, but is not limited thereto.

Therefore, higher tyrosinase enzyme inhibitory activity may indicate excellent skin pigmentation inhibition or skin whitening activity through the inhibition of melanin synthesis, but is not limited thereto.

In an embodiment of the present invention, the composition may inhibit the production of melanin, but is not limited thereto.

Human skin color is determined by the amounts of melanin, carotene, and hemoglobin, with melanin being the most decisive factor. Melanin is produced in pigment cells called melanocytes in the skin's basal layer and then transferred to surrounding keratinocytes, displaying human skin color. Excessive production of melanin is known to cause pigmentary disorders such as freckles and age spots. The enzyme tyrosinase in melanocytes, which plays a primary role in melanin synthesis, is particularly activated by ultraviolet rays. Therefore, exposure to a lot of sunlight may darken the skin, and inhibition of melanin synthesis and production mechanisms is essential for skin whitening or inhibiting skin pigmentation, but is not limited thereto.

In an embodiment of the present invention, the composition may decrease the expression of one or more melanin synthesis-related genes selected from the group consisting of tyrosinase, TRP-1, TRP-2, and MITF, but is not limited thereto.

In the present invention, tyrosinase related protein (TRP) is an intermembrane enzyme encoded by the TYRP1 gene in humans and may include TRP-1 and TRP-2.

TRP-1 is a melanocyte-specific gene product involved in melanin synthesis within melanosomes, and most TRP-1 may have oxidase activity for 5,6-dihydroxyindole-2-carboxylic acid (a melanin synthesis intermediate). TRP-1 is involved in the stabilization and catalytic activity regulation of tyrosinase protein, maintenance of melanosome structure, and may influence melanocyte proliferation and apoptosis. Additionally, melanocytes, derived from the neural crest, migrate to the upper ectoderm of the developing organism forming skin and hair, so TRP-1 may particularly affect melanin expression in the skin and hair of the organism, but is not limited thereto.

Microphthalmia-associated transcription factor (MITF) is a protein coded by the MITF gene in humans and can be denoted as class E basic helix-loop-helix protein 32 or bHLHe32, but is not limited thereto.

MITF is a basic helix-loop-helix leucine zipper transcription factor involved in the lineage specific pathway regulation of many types of cells, including melanocytes, osteoclasts, and mast cells. Because MITF exhibits lineage specificity, it may denote genes or traits found only in specific cell types, but is not limited thereto.

MITF may be involved in the rewiring of signaling cascades specifically necessary for the survival and physiological function of normal cell precursors, and in human subjects, MITF is known to regulate the expression of various genes essential for normal melanin synthesis in melanocytes. Mutations in MITF are known to cause diseases such as melanoma, Waardenburg syndrome, and Tietz syndrome, but are not limited thereto.

In the present invention, tyrosinase, TRP-1, TRP-2, and MITF may all be melanin synthesis-related genes, and as the expression of these genes or their expression is inhibited or suppressed, melanin synthesis may decrease, leading to the inhibition of skin pigmentation or improved skin whitening activity, but is not limited thereto.

In an embodiment of the present invention, the composition may increase the expression of at least one gene of (a), and decrease the expression of at least one gene of (b), but is not limited thereto:
(a) LC3-II, ATG5, and Beclin-1; and
(b) p62, and LC3-I.

In an embodiment of the present invention, the composition may inhibit autophagy inhibitors, but is not limited thereto.

In an embodiment of the present invention, the autophagy inhibitors may be at least one selected from the group consisting of wortmannin, bafilomycin, and 3-Methyladenine (3-MA), but is not limited thereto.

In the present invention, “autophagy” or “self-eating” is a process that involves the breakdown of intracellular proteins and organelles, including the transport of cytoplasmic material to lysosomes, representing lysosome-mediated catabolism that maintains cellular homeostasis through the degradation and recycling of cytoplasmic components and organelles, but is not limited thereto.

During the life span of a cell, dysfunctional organs and long-persisting proteins can accumulate and become targets for autophagy. When autophagy is activated, autophagosome membrane structures are formed by the atg12-atg5 complex and LC3 clustering, and the cytosolic form of LC3 (LC3-I) is cleaved into a membrane-bound form (LC3-II), maturing into an autophagosome that encloses the components for degradation. The autophagosome then fuses with lysosomes, leading to lysosomal degradation of intracellular components.

This system maintains cellular homeostasis and can induce either cell survival or cell death, but the specific mechanisms governing whether cell survival or death is induced remain unclear. Autophagy-induced cell death is classified as type II cell death and can be independent of caspase-dependent apoptosis, but is not limited thereto.

Autophagy may be associated with various pathological processes, including neurodegenerative diseases, cancer, and melanogenesis. In Parkinson's or Alzheimer's disease, microfibrils and amyloid plaques accumulate, and the induction of autophagy can provide a potential solution for the degradation of these accumulative components.

In cancer, autophagy can be associated with various stages. For example, in apoptosis-defective cancer cells, autophagy can be induced to promote cell death and regulate cell proliferation. In growing tumor cells, autophagy can be utilized for survival until the necessary components are provided by angiogenesis, as oxygen and nutrients are limited. In this case, autophagy should be inhibited to stop tumor cell survival. Some autophagy inducers simultaneously promote autophagy and apoptosis, which can synergistically kill cancer cells and eliminate addicted cells. Melanogenesis refers to the formation of melanin, a pigment found in the eyes, skin, and hair. Newly produced melanin from melanogenesis can exhibit different colors compared to existing melanin. During melanin production, melanosomes, distinct lysosome-related organelles, carry functional cargo, and melanosomes have been reported to be derived from autophagosomes, but are not limited thereto.

In this invention, LC3-II, ATG5, Beclin-1, p62, and LC3-I may be genes related to autophagy. For example, during autophagy activation, the expression or protein synthesis and activity of LC3-II, ATG5, and Beclin-1 may increase, while the expression or protein synthesis and activity of p62 and LC3-I may decrease, but are not limited thereto.

In the present invention, “autophagy inhibitors” may refer to substances that interfere with the action on any one or more of the series of mechanisms associated with autophagy activity, or proteins or cofactors such as enzymes and substrates involved in these processes, but are not limited thereto.

Alternatively, in the present invention, autophagy inhibitors may encompass substances that activate the action on any one or more of the series of mechanisms associated with autophagy inhibition, or proteins or cofactors such as enzymes and substrates involved in these processes, but are not limited thereto.

In the present invention, the term “autophagy inhibitors” may encompass the broadest definition as understood in the art.

Autophagy inhibitors in this invention may include wortmannin, bafilomycin, and 3-MA, but are not limited thereto. These inhibitors may include any generally or specifically known autophagy inhibitors in the field. Additionally, besides substances that inhibit autophagy activity itself, this invention may include substances that inhibit any one or more mechanisms among the series of processes related to autophagy, but are not limited thereto.

In the present invention, “food” means natural substances or products containing at least one nutrient, preferably being in a state ready for direct consumption after undergoing a certain degree of processing. Conventionally, it comprises functional foods, beverages, food additives, and beverage additives.

In the present invention, the “functional food” is a term synonymous with Food for Special Health Use (FoSHU), referring to foods that are medically and therapeutically effective, processed to efficiently manifest bio-regulatory functions besides providing nutrition. They may be prepared in forms such as tablets, capsules, pills, granules, powders, liquids, flakes, pastes, syrups, gels, jellies, bars, or films. “Functional” here means obtaining useful health effects, such as regulating nutrients or having physiological actions on the structure and function of the human body.

There are no specific limitations on the types of health functional foods in this invention. Specifically, examples of foods to which the composition of this invention can be added include dairy products including ice cream, various soups, beverages, tea, drink products, alcoholic beverages, and vitamin complexes. Particularly, it includes foods or food compositions designed to express sufficient bio-regulatory functions in the body, like body defense rhythm regulation, disease prevention and recovery, adding value to the conventional function of the relevant food for a specific purpose.

In the present invention, “food additives” pertain to substances used in the manufacture, processing, or preservation of food, added, mixed, infiltrated, or used by other methods. Like health functional foods, they must be harmless to the human body when consumed over a long period.

When using the composition of this invention as a food additive, the food additive may include the composition as is, or be used in conjunction with other foods or food ingredients, appropriately utilized according to conventional methods.

The amount of active ingredient mixed may be suitably determined according to the purpose of use (preventative, health, or therapeutic treatment). Generally, the composition of this invention may be added to the food or beverage ingredients in an amount of 15% by weight or less, or 10% by weight or less. However, for long-term consumption aimed at health and hygiene or health regulation, the amount may be below this range, and due to safety, the active ingredient may also be used in amounts above this range.

In the present invention, the composition may include a variety of food acceptable dietary supplements and may further include suitable carriers, excipients and diluents conventionally used in the preparation of food products.

Additionally, besides the aforementioned, this invention's composition may include various nutrients, vitamins, electrolytes, flavoring agents, coloring agents, pectic acids and their salts, alginic acids and their salts, organic acids, protective colloidal thickeners, pH adjusters, stabilizers, preservatives, glycerin, alcohol, carbonating agents used in carbonated drinks, etc. The composition may also include pulp for the production of natural fruit juices, fruit juice beverages, and vegetable drinks. These ingredients may be used independently or in combination. The proportion of these additives is not critical but typically, for every 100 parts by weight of this composition, 0.01 to 0.20 parts by weight is selected, although this is not limiting. The optimal and arbitrary amount may vary depending on the type and function of the product being utilized.

In this invention, there are no special restrictions on the types of food. Examples of foods to which the substance may be added include meats, sausages, bread, chocolate, candies, snacks, biscuits, pizza, ramen, other noodles, gum, dairy products including ice cream, various soups, beverages, tea, drink products, alcoholic beverages, and vitamin complexes, all of which may include all commonly understood health functional foods, but is not limited thereto.

Moreover, the composition according to this invention may be added to health drinks and may include various flavorings or natural carbohydrates as additional ingredients, similar to conventional beverages. The aforementioned natural carbohydrates may include monosaccharides such as glucose and fructose, disaccharides such as maltose and sucrose, polysaccharides such as dextrin and cyclodextrin, and sugar alcohols such as xylitol, sorbitol, and erythritol. As sweeteners, natural sweeteners like thaumatin and stevia extract, or synthetic sweeteners such as saccharin and aspartame may be used. The proportion of these natural carbohydrates may typically be about 0.01-0.20 g, or about 0.04-0.10 g per 100 mL of this composition, but this is not limiting and may include the general amounts added in the industry or the maximum range to enhance the efficacy of this composition. It may also include the optimal and arbitrary amount considering the synergistic effect with other added substances.

In the present invention, “alleviation” refers to all acts that enhance the skin condition due to the administration of the composition. More specifically, it means alleviating skin moisture, enhancing skin protective barriers, inhibiting cellular damage, reducing the number of wrinkles, or making the skin surface more elastic.

In the present invention, “skin condition improvement” pertains to the overall improvement of skin condition, as assessed through sensory evaluation of the skin, including brightness and transparency. More specifically, it encompasses inhibition of skin damage due to skin irritation, improvement through skin surface film enhancement, wrinkle improvement, or inhibition of skin aging.

In the present invention, “inhibition of skin damage due to skin irritation or improvement of the skin surface film” refers to the improvement of skin damage that occurs throughout the entire layer of the skin, including the dermis, caused by external or internal factors, particularly improvement of damage manifested in the epidermis of the skin.

In the present invention, “skin irritation” encompasses both physical and chemical stimuli. More specifically, physical stimuli include electrical, mechanical, thermal, light, sound, etc., and chemical stimuli include irritations caused by acids, alkalis, chemicals, etc. In this invention, skin irritation is preferably chemical stimulation, and more preferably, stimulation caused by chemicals contained in fine dust, but is not limited thereto.

The present invention provides a cosmetic composition for skin whitening or preventing or alleviating skin pigmentation, comprising procyanidin A2 and quercetin as active ingredients.

In the present invention, “cosmetic” may be understood to include all items that beautify the human appearance, but is not limited thereto, encompassing the broadest meaning used in the relevant industry.

The cosmetic composition of this invention may be used as an active ingredient in cosmetics or as a cosmetic additive, but is not limited thereto.

When used as a cosmetic additive in this invention, it may be utilized in the manufacture of cosmetic compositions for maintaining cleanliness of hands or feet. Examples include soaps (solid soap, liquid soap, foam soap, body soap, hand soap, etc.), cleansing foam, shampoos (hair shampoo, dry shampoo, etc.), but are not limited thereto.

The cosmetic composition of this invention may be manufactured in any formulation conventionally produced in the industry, for instance, as lotion (skin lotion), skin, skin softener, skin toner, astringent, solution, suspension, emulsion, paste, powder, gel, cream, hand cream, hand cleanser, lotion, milk lotion, moisture lotion, nourishing lotion, body lotion, body cleanser, powder, soap, surfactant-containing cleansing, oil, powder foundation, emulsion foundation, wax foundation, spray, and sheet, but is not limited thereto. More specifically, they may be formulated as softening water, nourishing water, nourishing cream, massage cream, moisture cream, essence, nourishing essence, eye cream, cleansing cream, cleansing foam, cleansing water, pack, spray or powder.

These formulations may be manufactured according to conventional methods in the field. The blending amount of additional ingredients such as moisturizers can be easily selected by those skilled in the art within a range that does not impair the purpose and effect of this invention.

In an embodiment of this invention, the cosmetic may be at least one selected from the group consisting of softening water, nourishing water, gel, essence, spray essence, emulsion, cream, lotion, powder, soap, surfactant-containing cleansing, oil, powder foundation, emulsion foundation, wax foundation, cleanser, bath powder, mask pack, massage cream, hand cream, sunscreen, body lotion, and body cleanser, but is not limited thereto.

In the present invention, the composition may be prepared in the form of common emulsion and solubilization formulations. Cosmetics in the form of emulsion formulation include nourishing water, creams, and essences, while those in the form of solubilization formulation include softening water. More specifically, the composition of this invention may be provided in the form of solutions, gels, solid or semi-solid anhydrous products, emulsions obtained by dispersing oil phase in water, suspensions, emulsions, microemulsions, microcapsules, mask packs, microgranules or ionic (liposome), non-ionic vesicle dispersants, creams, skins, lotions, powders, ointments, sprays, pastes, packs, cleansers, soaps, surfactant-containing cleansings, oils, powder foundations, bath powders, emulsion foundations, waxes, foundation, or concealer sticks. Additionally, they may also be manufactured in the form of foam or aerosol compositions containing compressed propellants.

When the composition is in the form of paste, cream, or gel, carrier components may include animal oils, vegetable oils, waxes, paraffins, starch, tragacanth, cellulose derivatives, polyethylene glycol, silicones, bentonite, silica, talc, zinc oxide, etc.

If the composition is in the form of powder or spray, carrier components may include lactose, talc, silica, aluminum hydroxide, calcium silicate, polyamide powder, and particularly for sprays, propellants such as chlorofluorohydrocarbon, propane/butane, dimethyl ether may be included.

If the composition is in the form of a solution or emulsion, carrier components may include solvents, solubilizers, emulsifiers, specifically water, ethanol, isopropanol, ethyl carbonate, ethyl acetate, benzyl alcohol, benzyl benzoate, propylene glycol, 1,3-butylene glycol, oil, glycerol fatty esters, polyethylene glycol, fatty acid esters of sorbitan, etc.

If the composition is in the form of a suspension, carrier components may include liquid diluents like water, ethanol, propylene glycol; suspending agents like ethoxylated isostearyl alcohol, polyoxyethylene sorbitol ester, polyoxyethylene sorbitan ester; and microcrystalline cellulose, aluminum metahydroxide, bentonite, agar, tragacanth, etc.

If the composition is a surfactant-containing cleansing, as carrier components, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamidobetaine, aliphatic alcohol, fatty acid glycerides, fatty acid diethanolamide, vegetable oils, lanolin derivatives, ethoxylated glycerol fatty acid esters, etc. may be included.

Additionally, other blendable components that may be added are not limited to the examples provided and may be blended as long as they do not impair the purpose and effect of this invention, preferably in a weight percentage of 0.01-10% of the total weight, more preferably 0.01-5%.

The cosmetic composition of this invention may further include functional additives and components commonly included in cosmetic compositions, in addition to the active ingredients disclosed in this specification. They may further include conventionally used purified water, thickeners, preservatives, stabilizers, solubilizers, surfactants, carriers, fragrances, or combinations thereof.

In the present invention, the carriers for the composition may be exemplified as alcohol, oil, surfactants, fatty acids, silicone oil, humectants, moisturizers, viscosity modifiers, emulsions, stabilizers, ultraviolet light scattering agents, ultraviolet light absorbers, coloring agents, fragrances, etc. The compounds/compositions that may be used as alcohol, oil, surfactants, fatty acids, silicone oil, humectants, moisturizers, viscosity modifiers, emulsions, stabilizers, ultraviolet light scattering agents, ultraviolet light absorbers, coloring agents, fragrances are already known in the art, thus an artisan can select and use an appropriate substance/composition. Furthermore, the cosmetic composition may include, as needed, sunscreens, antioxidants (such as butylated hydroxyanisole, propyl gallate, erythorbic acid, tocopheryl acetate, butylated hydroxytoluene), preservatives (such as methylparaben, butylparaben, propylparaben, phenoxyethanol, imidazolidinyl urea, chlorphenesin), coloring agents, pH adjusters (such as triethanolamine, citric acid, sodium citrate, malic acid, sodium malate, fumaric acid, sodium fumarate, succinic acid, sodium succinate, sodium hydroxide, monosodium phosphate), moisturizers (such as glycerin, sorbitol, propylene glycol, butylene glycol, hexylene glycol, diglycerin, betaine, glycereth-26, methyl gluceth-20), and lubricants.

In the present invention, the composition may further include at least one known active ingredient having a skin condition alleviating effect, and furthermore, at least one substance commonly used in cosmetic compositions. Specifically, fatty materials, organic solvents, solubilizers, thickeners, and gelling agents, softeners, antioxidants, suspending agents, stabilizers, foaming agents, fragrances, surfactants, water, ionic or nonionic emulsifiers, fillers, metal ion blockers and chelating agents, preservatives, vitamins, blockers, humectants, essential oils, dyes, pigments, hydrophilic or lipophilic active agents, lipid vesicles, or any other cosmetic or dermatological auxiliary agents typically used in cosmetics or dermatology may also be additionally contained. Moreover, these ingredients may be incorporated in amounts generally used in the field of skin science.

In the present invention, the composition may further include a composition selected from the group consisting of water-soluble vitamins, fat-soluble vitamins, high molecular peptides, high molecular polysaccharides, and sphingolipids.

Water-soluble vitamins may be any that may be incorporated in cosmetics, for example, vitamin B1, B2, B6, pyridoxine, pyridoxine hydrochloride, vitamin B12, pantothenic acid, nicotinic acid, nicotinamide, folic acid, vitamin C, vitamin H, and their salts (such as thiamine hydrochloride, sodium ascorbate) or derivatives (such as sodium ascorbate-2-phosphate, magnesium ascorbate-2-phosphate) are also included in water-soluble vitamins used in this invention. Water-soluble vitamins may be obtained by conventional methods such as microbial conversion, purification from microbial cultures, enzymatic methods, or chemical synthesis.

Fat-soluble vitamins may be any that may be incorporated in cosmetics, for example, vitamin A, carotene, vitamin D2, D3, vitamin E (d1-alpha-tocopherol, d-alpha-tocopherol), and their derivatives (such as ascorbyl palmitate, ascorbyl stearate, diascorbyl palmitate, acetyl dl-alpha-tocopherol, nicotinic acid, dl-alpha tocopherol (vitamin E), dl-pantothenyl alcohol, D-pantothenyl alcohol, pantothenyl ethyl ether) are also included in fat-soluble vitamins used in this invention. Fat-soluble vitamins may be obtained by conventional methods such as microbial conversion, purification from microbial cultures, enzymatic methods, or chemical synthesis.

The polymer peptides may be any substance that is blendable with cosmetics, and examples thereof may include collagen, hydrolyzed collagen, gelatin, elastin, hydrolyzed elastin, and keratin. The polymer peptides may be purified and obtained by any conventional method such as purification from microbial cultures, enzymatic methods, or chemical synthesis, or may generally be used by being purified from natural substances such as the dermis of a pig, a cow, or the like and silk fiber of silkworms.

The polymeric polysaccharides may be any substance that is blendable with cosmetics, and examples thereof may include hydroxyethyl cellulose, xanthan gum, sodium hyaluronate, and chondroitin sulfate or salts thereof (sodium salts). For example, chondroitin sulfate or salts thereof may generally be purified from mammals or fish and used.

The sphingolipids may be any substance that is blendable with cosmetics, and examples thereof may include ceramide, phytosphingosine, and sphingoglycolipid. The sphingolipids may be purified, by a conventional method, from mammals, fish, shellfish, yeast, or plants, or may be obtained by chemical synthesis.

The cosmetic composition of the present invention may include, as necessary, other ingredients mixed in conventional cosmetics along with the above essential ingredients. Examples of additional ingredients to be mixed may include lipid components, humectants, emollients, surfactants, organic and inorganic pigments, organic powder, ultraviolet light absorbers, preservatives, sanitizers, antioxidants, plant extracts, pH adjusters, alcohol, pigments, flavors, blood circulation promoters, cooling agents, anti-diaphoretics, and purified water.

The lipid components may include, for example, ester lipids, hydrocarbon lipids, silicone lipids, fluorine lipids, animal fats, vegetable oils, or the like.

The ester lipids may include, for example, glyceryl tri 2-ethylhexanoate, cetyl 2-ethylhexanoate, isopropyl myristate, butyl myristate, isopropyl palmitate, ethyl stearate, octyl palmitate, isocetyl isostearate, butyl stearate, ethyl linolate, isopropyl linolate, ethyl oleate, isocetyl myristate, isostearyl myristate, isostearyl palmitate, octyldodecyl myristate, isocetyl isostearate, diethyl sebacate, diisopropyl adipate, isoalkyl neopentanate, tri(capryl, capric acid)glyceryl, trimethylolpropane tri 2-ethylhexanoate, trimethylolpropane triisostearate, pentaerythritol tetra 2-ethylhexanoate, cetyl caprylate, decyl laurate, hexyl laurate, decyl myristate, myristyl myristate, cetyl myristate, stearyl stearate, decyl oleate, cetyl ricinoleate, isostearyl laurate, isotridecyl myristate, isocetyl palmitate, octyl stearate, isocetyl stearate, isodecyl oleate, octyldodecyl oleate, octyldodecyl linolate, isopropyl isostearate, cetostearyl 2-ethylhexanoate, stearyl 2-ethylhexanoate, hexyl isostearate, ethyleneglycol dioctanoate, ethyleneglycol dioleate, propyleneglycol dicaprinate, propyleneglycol di(caprylate, caprinate), propyleneglycol dicaprylate, neopentylglycol dicaprinate, neopentylglycol dioctanoate, glyceryl tricaprylate, glyceryl triundecylate, glyceryl triisopalmitate, glyceryl triisostearate, octyldodecyl neopentanoate, isostearyl octanoate, octyl isononanoate, hexyldecyl neodecanoate, octyldodecyl neodecanoate, isocetyl isostearate, isostearyl isostearate, octyldecyl isostearate, polyglycerin ester oleate, polyglycerin ester isostearate, triisocetyl citrate, triisoalkyl citrate, triisooctyl citrate, lauryl lactate, myristyl lactate, cetyl lactate, octyldecyl lactate, triethyl citrate, acetyltriethyl citrate, acetyltributyl citrate, trioctyl citrate, diisostearyl malate, 2-ethylhexyl hydroxystearate, di 2-ethylhexyl succinate, diisobutyl adipate, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoyl hydroxystearate, stearoyl 12-stearoyl hydroxystearate, isostearyl 12-stearoyl hydroxystearate, and the like.

The hydrocarbon lipids may include, for example, squalene, liquid paraffin, alpha-olefin oligomers, isoparaffin, ceresine, paraffin, liquid isoparaffin, polybutene, microcrystalline wax, Vaseline, and the like.

The silicone lipids may include, for example, polymethyl silicon, methylphenyl silicon, methyl cyclopolysiloxane, octamethyl polysiloxane, decamethyl polysiloxane, dodecamethyl cyclosiloxane, dimethylsiloxane/methylcetyloxysiloxane copolymers, dimethylsiloxane/methylstearoxysiloxane copolymers, alkyl-modified silicon oil, amino-modified silicon oil, and the like.

The fluorine lipids may include perfluoropolyether and the like.

The animal or vegetable oil may include avocado oil, almond oil, olive oil, sesame oil, rice bran oil, safflower oil, soybean oil, corn oil, rape flower oil, apricot kernel oil, palm kernel oil, palm oil, castor oil, sunflower oil, grape seed oil, cotton seed oil, coconut oil, tallow nut oil, wheat germ oil, rice germ oil, Shea butter, evening primrose oil, macadamia nut oil, meadow foam seed oil, yolk oil, beef tallow, hemp seed oil, mink oil, orange roughy oil, jojoba oil, candelilla wax, carnauba wax, liquid lanolin, dehydrated castor oil, and the like.

The humectant may include water-soluble low molecular humectants, oil-soluble molecular humectants, water-soluble polymers, oil-soluble polymers, and the like.

The water-soluble low molecular humectants may include serine, glutamine, sorbitol, mannitol, pyrrolidone-sodium carboxylate, glycerin, propylene glycol, 1,3-butylene glycol, ethylene glycol, polyethylene glycol B (degree of polymerization: n=2 or higher), polypropylene glycol (degree of polymerization: n=2 or higher), polyglycerin B (degree of polymerization: n=2 or higher), lactic acid, lactates, and the like.

The oil-soluble low molecular humectants may include cholesterol, cholesterol ester, and the like.

The water-soluble polymers may include carboxyvinyl polymers, polyasparaginic acid salts, tragacanth, xanthan gum, methyl cellulose, hydroxymethyl cellulose, hydroxylethyl cellulose, hydroxypropyl cellulose, carboxymethyl cellulose, water-soluble chitin, chitosan, dextrin, and the like.

The oil-soluble polymers may include, for example, polyvinyl pyrrolidone/eicosen copolymers, polyvinyl pyrrolidone/hexadecene copolymers, nitrocellulose, dextrin fatty acid ester, silicone polymers, and the like.

The emollients may include, for example, long chain cholesterylester acyl glutamate, cholesteryl hydroxystearate, 12-hydroxystearic acid, stearic acid, rosin acid, lanolin fatty acid cholesteryl ester, and the like.

The surfactants may include, for example, non-ionic surfactants, anionic surfactants, cationic surfactants, amphoteric surfactants, and the like.

The non-ionic surfactants may include self-emulsion type glycerin monostearate, propyleneglycol fatty acid ester, glycerin fatty acid ester, polyglycerin fatty acid ester, sorbitan fatty acid ester, polyoxyethylene (POE) sorbitan fatty acid ester, POE sorbit fatty acid ester, POE glycerin fatty acid ester, POE alkylethers, POE fatty acid ester, POE dehydrated castor oil, POE castor oil, polyoxyethylene/polyoxypropylene (POE/POP) copolymers, POE/POP alkylethers, polyether-modified silicone, alkanolamide laurate, alkylamine oxide, hydrated soy phospholipids, and the like.

The anionic surfactants may include fatty acid soap, α-acylsulfonate, alkyl sulfonates, alkylallyl sulfonates, alkylnaphthalene sulfonates, alkyl sulfates, POE alkylether sulfates, alkylamide sulfates, alkyl phosphates, POE alkyl phosphates, alkylamide phosphates, alkyloyl alkyltaurin salts, N-acylamino acid salts, POE alkylether carboxylates, alkyl sulfosuccinates, sodium alkyl sulfoacetates, acylated hydrolyzed collagen peptide salts, perfluoroalkyl ester phosphates, and the like.

The cationic surfactants may include, for example, alkyltrimethylammonium chloride, stearyltrimethylammonium chloride, steraryltrimethylammonium bromide, cetostearyl trimethylammonium chloride, distearyl dimethylammonium chloride, stearylaryl dimethylbenzylammonium chloride, behenyltrimethylammonium bromide, benzalkonium chloride, diethylaminoethylamide stearate, dimethylaminopropylamide stearate, quaternary ammonium salts of lanolin derivatives, and the like.

The amphoteric surfactants may include carboxybetaine, amidebetaine, sulfobetaine, hydroxysulfobetaine, amidesulfobetaine, phosphobetaine, aminocarboxylate, imidazoline derivatives, amideamine-based amphoteric surfactants, and the like.

The organic and inorganic pigments may include: inorganic pigments such as silicic acid, anhydrous silicic acid, magnesium silicate, talc, sericite, mica, kaolin, bengala, clay, bentonite, titanium dioxide-coated mica, bismuth oxychloride, zirconium oxide, magnesium oxide, zinc oxide, titanium oxide, aluminum oxide, calcium sulfate, barium sulfate, magnesium sulfate, calcium carbonate, magnesium carbonate, iron oxide, ultramarine, chromium oxide, chromium hydroxide, calamine, and combinations thereof; organic pigments such as polyamide, polyester, polypropylene, polystyrene, polyurethane, vinyl resin, urea resin, phenol resin, fluorine resin, silicon resin, acryl resin, melamine resin, epoxy resin, polycarbonate resin, divinyl benzene/styrene copolymers, silk powder, cellulose, CI pigment yellow, and CI pigment orange; and composite pigments of inorganic and organic pigments.

The organic powder may include: metallic soap such as calcium stearate; metal salts of alkyl phosphoric acid such as zinc sodium cetylate, zinc laurylate, and calcium laurylate; polymetallic salts of acylamino acid such as calcium N-lauroyl-beta-alanine, zinc N-lauroyl-beta-alanine, and calcium N-lauroylglycine; polymetallic salts of amide sulfonates such as calcium N-lauroyl-taurine and calcium N-palmitoyl-taurine; N-acyl alkaline amino acids such as N-epsilon-lauroyl-L-lysine, N-epsilon-palmitoyl lysine, N-α-palmitoylol nitin, N-α-lauroyl arginine, and N-α-dehydrated tallow fatty acid acyl arginine; N-acyl polypeptides such as N-lauroyl glycylglycine: α-amino fatty acids such as α-aminocaprylic acid and a-aminolauric acid; polyethylene; polypropylene; nylon; polymethylmethacrylate; polystyrene; divinylbenzene/styrene copolymers; ethylene tetrafluoride; and the like.

The ultraviolet light absorbers may include para-aminobenzoic acid, ethyl para-aminobenzoate, amyl para-aminobenzoate, octyl 1 para-aminobenzoate, ethyleneglycol salicylate, phenyl salicylate, octyl salcylate, benzyl salicylate, butylphenyl salicylate, homomentyl salicylate, benzyl cinnamate, para-methoxycinnamic acid-2-ethoxylethyl, octyl paramethoxycinnamate, mono-2-ethylhexaneglyceryl diparamethoxycinnamate, isopropyl paramethoxycinnamate, diisopropyl/diisopropyl cinnamic acid ester mixtures, urocanic acid, ethyl urocanate, hydroxymethoxy benzophenone, hydroxymethoxybenzophenone sulfonic acid and salts thereof, dihydroxymethoxy benzophenone, sodium dihydroxymethoxy benzophenone disulfonate, dihydroxy benzophenone, tetrahydroxy benzophenone, 4-tert-butyl-4′-methoxydibenzoylmethane, 2,4,6-trianilino-p-(carbo-2′-ethylhexyl-1′-oxy)-1,3,5-triazine, 2-(2-hydroxy-5-methylphenyl)benzotriazole, and the like.

The sanitizers may include hinokitiol, trichloric acid, trichlorohydroxydiphenylether, chlorohexidine gluconate, phenoxyethanol, resorcine, isopropylmethylphenol, azulene, salicylic acid, zinc pyrithione, benzalkonium chloride, light sensitive element No. 301, sodium mononitroguaiacol, undecylenic acid, and the like.

The antioxidants may include butylhydroxyanisole, propyl gallate, elisorbic acid, and the like.

The pH adjusters may include citric acid, sodium citrate, malic acid, sodium malate, fumaric acid, sodium fumarate, succinic acid, sodium succinate, sodium hydroxide, sodium monohydrophosphate, and the like.

The alcohols may include higher alcohols such as cetyl alcohol.

In the present invention, the components included in the cosmetic composition comprise, in addition to the composition as active ingredients, the components typically used in cosmetic compositions. These include conventional auxiliaries such as antioxidants, stabilizers, solubilizers, vitamins, pigments, and fragrances, and also include a carrier. Moreover, the cosmetic composition may further include a skin absorption enhancer to enhance its effects, but is not limited thereto.

The present invention provides a quasi-drug composition for skin whitening or for preventing or alleviating skin pigmentation, comprising procyanidin A2 and quercetin as active ingredients.

In an embodiment of the present invention, the quasi-drug composition may be for non-oral administration, but is not limited thereto.

In this specification, the term “quasi-drug” may refer to preparations used for the prevention of infectious diseases through sterilization, insecticide, and similar uses, as defined in Article 2, Paragraph 7, Item (c) of the Pharmaceutical Affairs Act. For example, under the Pharmaceutical Affairs Act, quasi-drugs exclude products used for the purposes of medicine, and include textile and rubber products used for the treatment or prevention of diseases in humans or animals, products that have a mild effect on the human body or do not act directly, and are not devices or machinery, and similar products, and include sterilizers and insecticides for preventing infectious diseases.

Furthermore, the quasi-drug may include topical skin agents and personal hygiene products.

The term “skin” here may include all skin areas of the body, including face, hands, arms, legs, feet, chest, abdomen, back, buttocks, and scalp, but is not limited thereto.

The types and formulations of the quasi-drug composition of the present invention are not particularly limited and may include bandages, gauze, absorbent cotton, adhesive plasters, disinfectants, shower foams, gargles, wet tissues, cleaning soaps, hand washes, humidifier fillers, masks, or filter fillers, among others.

When the composition of the present invention is included in a quasi-drug for the purpose of alleviating skin conditions, the composition may be used as is or in combination with other quasi-drug components, and may be suitably used according to conventional methods. The mixing amount of the active ingredient may be appropriately determined according to the purpose of use, and the quasi-drug composition of the present invention may include 0.01 to 20 weight % of microbes, their solutions, culture fluids, or a mixture thereof, relative to the total weight of the composition.

In the present invention, the topical skin agents may include creams, gels, ointments, skin emulsions, skin suspensions, transdermal patches, lotions, or combinations thereof. These topical skin agents typically include ingredients used in skin external preparations such as cosmetics or pharmaceuticals, for example, aqueous components, oily components, powdery components, alcohols, moisturizers, thickeners, ultraviolet light absorbers, whitening agents, preservatives, antioxidants, surfactants, fragrances, dyes, various skin nutrients, or combinations thereof, and may be appropriately mixed as required. The topical skin agents may also be appropriately mixed with metal chelating agents such as edetate disodium, edetate trisodium, sodium citrate, sodium polyphosphate, sodium metaphosphate, gluconate; pharmacologically active substances like caffeine, tannin, verapamil, licorice extract, glabridin, hot-water extract of the fruit of karin, various herbal medicines, acetate tocopherol, glycyrrhizic acid, tranexamic acid and its derivatives or salts; vitamin C, magnesium ascorbyl phosphate, ascorbyl glucoside, arbutin, kojic acid; and sugars like glucose, fructose, trehalose.

The topical skin agent composition according to the present invention may contain 0.00001 to 80 weight % of microbes, their solutions, culture fluids, or mixtures thereof, based on the total weight of the composition. However, this is not limiting, as the addition amount and types of ingredients may include all amounts, mixing ratios, and materials generally added in the field.

The present invention provides a pharmaceutical composition for preventing or treating skin pigmentation disorders, comprising procyanidin A2 and quercetin as active ingredients.

In an embodiment of the present invention, the skin pigmentation disorders may be one or more selected from the group consisting of melasma, freckles, lentigo, seborrheic keratosis, mole, café-au-lait spots, nevus of Ota, blue nevus, hyperpigmentation spots, drug-induced hyperpigmentation, gravidic chloasma, and post-inflammatory hyperpigmentation due to wounds or dermatitis, but is not limited thereto.

The pharmaceutical composition according to the present invention may further include appropriate carriers, excipients, and diluents typically used in the preparation of pharmaceutical compositions. The excipients may be selected from the group consisting of diluents, binders, disintegrants, lubricants, adsorbents, moisturizers, film coating materials, and controlled-release additives.

The pharmaceutical composition according to the present invention may be used by being formulated, according to commonly used methods, into a form such as powders, granules, sustained-release-type granules, enteric granules, liquids, eye drops, elixirs, emulsions, suspensions, spirits, troches, aromatic water, lemonades, tablets, sustained-release-type tablets, enteric tablets, sublingual tablets, hard capsules, soft capsules, sustained-release-type capsules, enteric capsules, pills, tinctures, soft extracts, dry extracts, fluid extracts, injections, capsules, perfusates, or a preparation for external use, such as plasters, lotions, pastes, sprays, inhalants, patches, sterile injectable solutions, or aerosols. The preparation for external use may have a formulation such as creams, gels, patches, sprays, ointments, plasters, lotions, liniments, pastes, or cataplasmas.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxy benzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soy bean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, fragrances, colorants, preservatives, stabilizers, suspending agents, emulsifiers, viscous agents, or the like may be used.

In emulsions according to the present invention, purified water may be used, and as necessary, emulsifiers, preservatives, stabilizers, fragrances, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, surfactants, preservatives, stabilizers, colorants, and fragrances may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter+cholesterol, lecithin, lanette wax, glycerol monostearate, Tween or span, imhausen, monolan (propylene glycol monostearate), glycerin, Adeps solidus, buytyrum Tego-G, cebes Pharma 16, hexalide base 95, cotomar, Hydrokote SP, S-70-XXA, S-70-XX75 (S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

The pharmaceutical composition according to the present invention is administered in a pharmaceutically effective amount. In the present invention, “the pharmaceutically effective amount” refers to an amount sufficient to treat diseases at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this may be easily determined by those of ordinary skill in the art.

The pharmaceutical composition of the present invention may be administered to a subject via various routes. All administration methods can be predicted, and the pharmaceutical composition may be administered via, for example, oral administration, subcutaneous injection, intraperitoneal injection, intravenous injection, intramuscular injection, intrathecal (space around the spinal cord) injection, sublingual administration, administration via the buccal mucosa, intrarectal insertion, intravaginal insertion, ocular administration, intra-aural administration, intranasal administration, inhalation, spraying via the mouth or nose, transdermal administration, percutaneous administration, or the like.

The pharmaceutical composition of the present invention is determined depending on the type of a drug, which is an active ingredient, along with various related factors such as a disease to be treated, administration route, the age, gender, and body weight of a patient, and the severity of diseases.

As used herein, the “subject” refers to a subject in need of treatment of a disease, and more specifically, refers to a mammal such as a human or a non-human primate, a mouse, a rat, a dog, a cat, a horse, and a cow, but the present invention is not limited thereto.

As used herein, the “administration” refers to providing a subject with a predetermined composition of the present invention by using an arbitrary appropriate method. The term “prevention” as used herein means all actions that inhibit or delay the onset of a target disease. The term “treatment” as used herein means all actions that alleviate or beneficially change a target disease and abnormal metabolic symptoms caused thereby via administration of the pharmaceutical composition according to the present invention. The term “alleviation” as used herein means all actions that reduce the degree of parameters related to a target disease, e.g., symptoms via administration of the composition according to the present invention.

Hereinafter, preferable examples are presented to aid in the understanding of the present invention. However, these examples are provided merely for easier understanding of the present invention, and the content of the present invention is not limited by these examples.

### EXAMPLES

### Example 1. Analysis of Tyrosinase Inhibitory Activity of Procyanidin A2 and Quercetin

The present invention conducted an analysis of tyrosinase inhibitory activity to confirm the melanin production inhibitory capacity of procyanidin A2 and quercetin, respectively. In the reaction mixture, 0.5 ml of 0.175 M sodium phosphate buffer (pH 6.8), 0.2 ml of a substrate solution containing 10 mM L-DOPA, and 0.1 ml of the sample solution were mixed, followed by the addition of 0.2 ml of mushroom tyrosinase (110 unit/ml). The reaction was carried out for 2 minutes at 25° C., and the DOPA chrome produced in the reaction mixture was measured at 475 nm. Tyrosinase inhibitory activity was expressed as the percentage reduction in absorbance compared to the control group without sample solution.

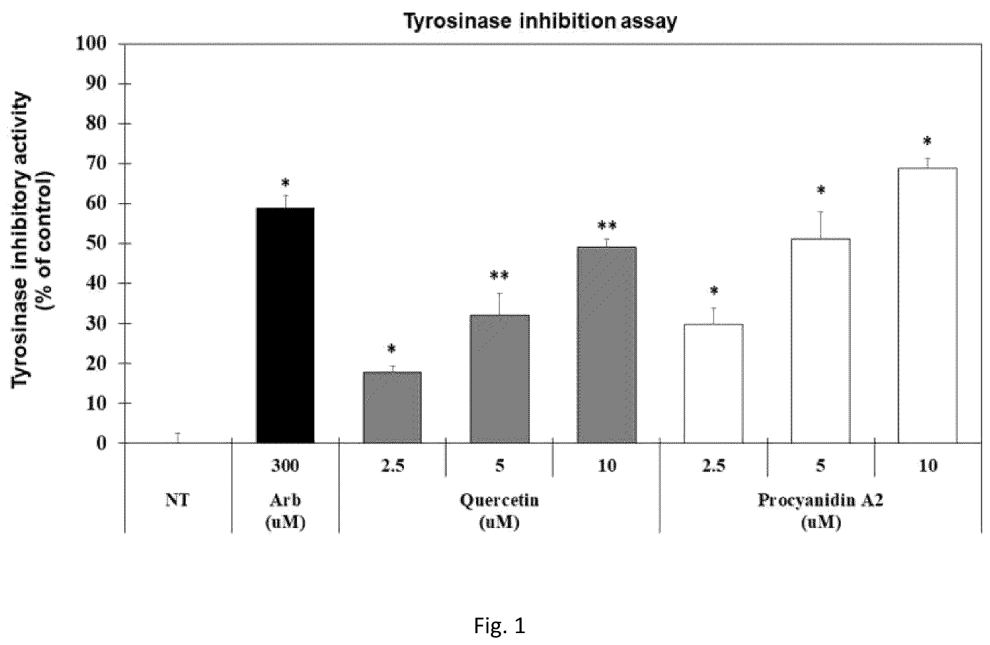

As shown in FIG. 1, quercetin showed inhibitory activities of 17.65%, 32.08%, and 49.44% at concentrations of 2.5, 5, and 10 μM, respectively. Under the same conditions, procyanidin A2 showed 29.82%, 51.12%, and 68.81%, respectively, indicating that both compounds exhibit concentration-dependent tyrosinase inhibitory activity. This was more effective than the positive control arbutin, which showed 58.79% inhibition at 300 μM concentration.

### Example 2. Determination of Optimal Ratio of Procyanidin A2 and Quercetin Combination Based on Tyrosinase Inhibitory Activity

Following Example 1, the tyrosinase inhibitory activity of the combination (hereinafter referred to as “the combination”) comprising procyanidin A2 and quercetin, which were confirmed to exhibit tyrosinase inhibitory activity, was evaluated to determine the optimal ratio of procyanidin A2 and quercetin.

TABLE 1

| | | Procyanidin | | Quercetin | | Procyanidin | | | | |
|---|---|---|---|---|---|---|---|---|---|---|
| Group | Untreated | A2 (A2) | | (QC) | | A2:Quercetin (AQ) | | | | |
| Concentration | — | 2.5 | 5 | 2.5 | 5 | 2.5:5 | 5:2.5 | 5:5 | 5:10 | 10:5 |
| (μM) | | | | | | | | | | |

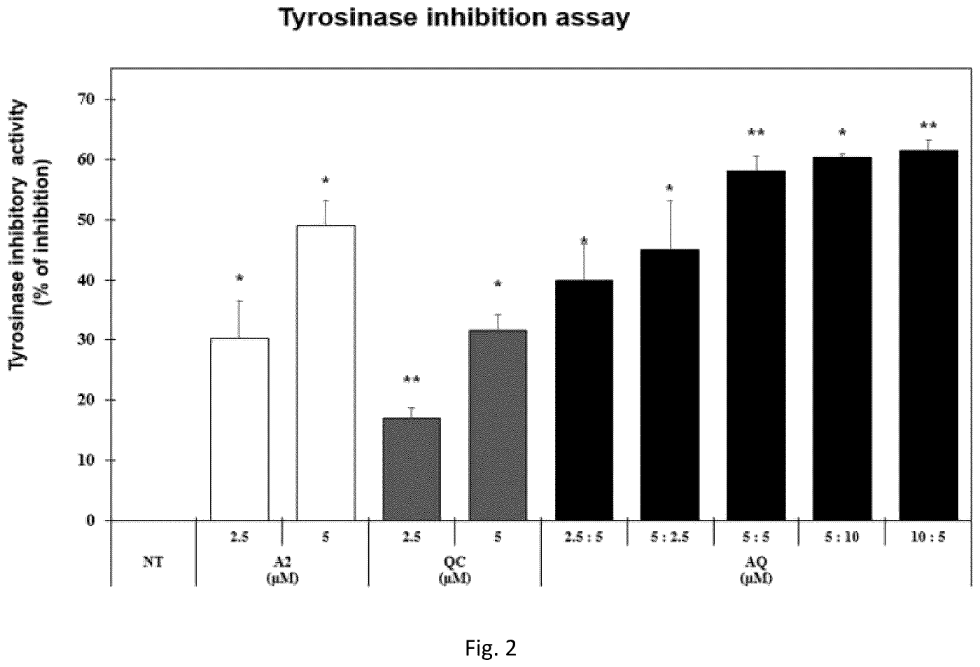

After setting the control and experimental groups as shown in Table 1, the tyrosinase inhibitory activity experiment was conducted in the same manner as Example 1, and the inhibitory activity (%) for each group was confirmed as shown in Tables 2, 3 and FIG. 2.

First, the inhibitory activities of procyanidin A2 and quercetin each increased proportionally to the concentration. Additionally, the inhibitory activity of 5 μM quercetin appeared to be at a similar level to that of 2.5 μM procyanidin A2, indicating that procyanidin A2 has a more superior tyrosinase inhibitory effect compared to quercetin.

TABLE 2

| Group | Concentration (μM) | Inhibitory Activity (%) |
|---|---|---|
| Procyanidin A2 (A2) | 2.5 | 30.27 |
| | 5 | 49.04 |
| Quercetin (QC) | 2.5 | 17.05 |
| | 5 | 31.61 |

For the combination comprising procyanidin A2 and quercetin, it was found that the tyrosinase inhibitory activity was superior when each compound was mixed in similar ratios. Specifically, the inhibitory activities of combinations mixed at ratios of 1:1, 1:2, and 2:1 were significantly higher compared to those mixed at ratios of 5:1 and 1:5. However, as the inhibitory activities of combinations mixed at ratios of 1:1, 1:2, and 2:1 at 5 μM concentration were similar, and there was no significant difference in tyrosinase inhibitory activity when using a ratio exceeding 1:1 (*p>0.05, **p>0.01), it was determined that the most efficient tyrosinase inhibition ratio is 1:1.

Additionally, consistent with the individual compound inhibition analysis results, combinations with a higher ratio of procyanidin A2 exhibited superior tyrosinase inhibitory effects compared to those with a lower ratio.

TABLE 3

| Procyanidin A2:Quercetin (AQ) (μM) | Inhibitory Activity (%) |
|---|---|
| 2.5:5 | 39.84 |
| 5:2.5 | 45.02 |
| 5:5 | 58.05 |
| 5:10 | 60.36 |
| 10:5 | 61.49 |

Based on the results, all subsequent experiments used the combination of procyanidin A2 and quercetin mixed at a 1:1 ratio.

### Example 3. Comparison of Cell Toxicity Between Procyanidin A2, Quercetin, and their Combination

To examine the cell toxicity of procyanidin A2, quercetin, and their combination, a cell viability analysis was conducted. Specifically, cell survival and cytotoxicity were measured using the 3-(4,5-dimethylthiazol-2-yl)-2,5-diphenyltetrazoliumbromide (MTT) Assay. MNT-1 cells were seeded in a 96 well plate at 1×10⁵ cells/well and cultured for 24 hours in a 37° C., 5% CO₂ incubator. After that, each compound was treated according to Table 4 and cultured for 72 hours. Then, MTT solution at a final concentration of 0.5 mg/ml was added and reacted for 4 hours at 37° C. The media containing MTT solution was removed, and 100 μl of DMSO was treated in each well to dissolve the formazan precipitate formed in reaction with the living cells. Absorbance was measured at 550 nm using an ELISA reader (CLARIOstar Plus, BMG LABTECH, Germany).

TABLE 4

| | | Procyanidin | | | | | | | | | | |
|---|---|---|---|---|---|---|---|---|---|---|---|---|
| Group | Untreated | A2 (A2) | | | | | Quercetin (AQ) | | | | | AQ |
| Concentration | — | 5 | 10 | 25 | 50 | 100 | 5 | 10 | 25 | 50 | 100 | 20:20 |
| (μM) or Ratio | | | | | | | | | | | | |

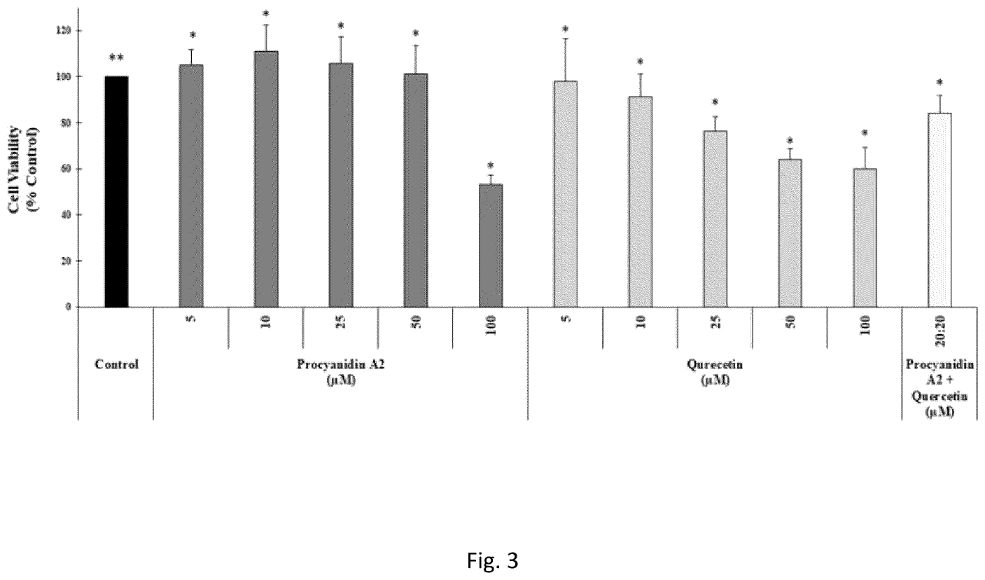

As per the results shown in FIG. 3, procyanidin A2 showed toxicity at 100 μM but no cytotoxicity at concentrations below 50 μM, and quercetin showed no cytotoxicity at approximately 25 μM and below. The combination, mixed equally at 20 μM of each compound, also showed no cytotoxicity.

### Example 4. Analysis of Melanin Production with Treatment of Procyanidin A2, Quercetin, and Their Combination

The melanin inhibitory effects of each substance were confirmed through an analysis of melanin production following treatment with procyanidin A2, quercetin, and their combination. Specifically, MNT-1 cells (human-derived melanoma skin cells) were treated for 72 hours with procyanidin A2, quercetin, and the combination at the maximum concentration without cytotoxicity according to Table 5. The negative control group was untreated, and arbutin, a tyrosinase inhibitor, was used as the positive control. Afterward, the media was removed, the cell bodies were dissolved with 1 N NaOH, heated at 95° C. for 5 minutes, and the absorbance was measured at 490 nm using a microplate reader (Molecular Devices, Sunnyvale, CA, USA).

TABLE 5

| | Negative | Positive | | | |
|---|---|---|---|---|---|
| | Control | Control | A2 | QC | AQ |
| Group | (Untreated) | (Arbutin, mM) | (μM) | (μM) | (μM) |
| Concentration | — | 1 | 50 | 20 | 20:20 |

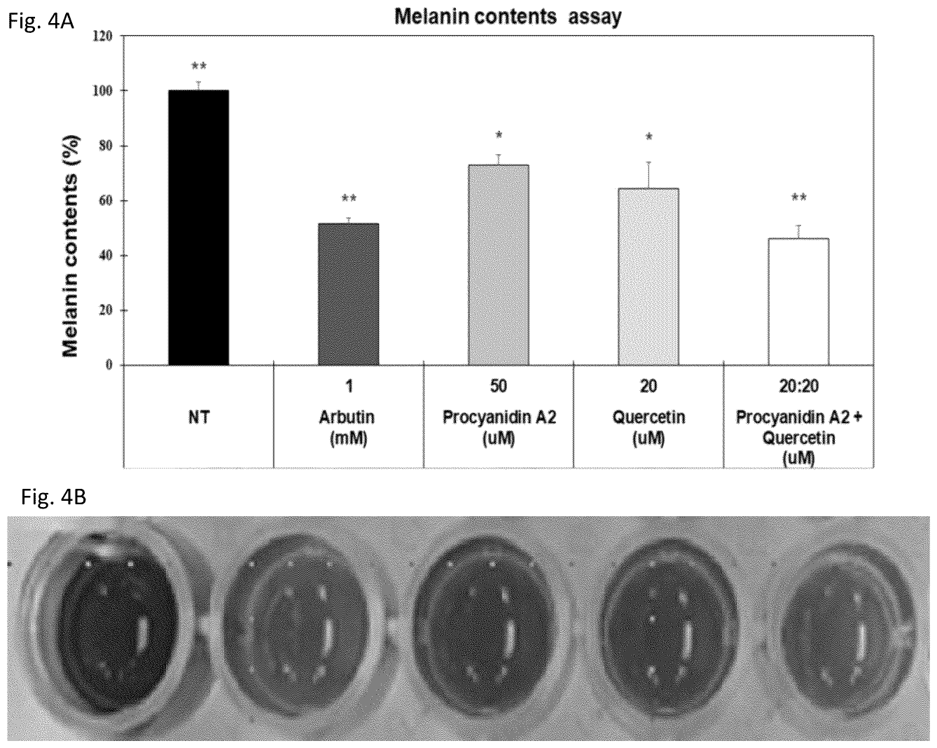

As a result, FIGS. 4A and 4B showed that when the absorbance was calculated and the amount of melanin produced in the NT (non-treatment group) was set to 100%, when treated with arbutin, melanin production was reduced by about 57.1%, but when treated with procyanidin A2 and quercetin, it was reduced to 72.3% and 64.5%. Respectively, indicating that single compounds were less effective in inhibiting tyrosinase compared to arbutin. However, treatment with the combination reduced tyrosinase activity to 52.2%, demonstrating a superior reduction effect compared to all control and experimental groups, and this effect was also visually observable.

These results indicate that the whitening effect of the present invention's combination is not only superior to the control group but also significantly effective at a much lower dose (about 1/25) compared to arbutin, the tyrosinase inhibitor, confirming a significant synergistic effect in inhibiting tyrosinase activity with the combination of both compounds.

### Example 5. Analysis of Melanin Synthesis-Related Protein Expression Following Treatment with Procyanidin A2, Quercetin, and Their Combination

MITF (microphthalmia-associated transcription factor) is a b-HLH-Zip (basic helix-loop-helix-leucine zipper) transcription factor expressed in various tissues. MITF mediates the differentiation effect of melanocyte-stimulating hormone (α-MSH) by regulating enzymes essential for melanin production in differentiated melanocytes. Tyrosinase, TRP-1, and TRP-2, expressed by MITF, are involved in melanin pigment production mediated by tyrosine. Therefore, the expression levels of these melanin synthesis-related proteins were analyzed to confirm whether the present invention's procyanidin A2, quercetin, and their combination exhibit melanin synthesis inhibitory effects.

Specifically, after treating with procyanidin A2, quercetin, and the combination, MNT-1 cells were washed twice in ice-cold PBS and lysed in ice-cold RIPA buffer (Sigma-Aldrich). Protein concentration was measured using a Bicinchoninic acid (BCA) protein assay kit (Pierce, Rockford, IL, USA), and 35 μg of protein was separated using sodium dodecyl sulfate-polyacrylamide gel electrophoresis (10%) and transferred to a nitrocellulose membrane (Whatman, Dassel, Germany). The membrane was blocked in 5% non-fat milk in TBST buffer (1:5000) and then treated with primary (1:1000) and secondary antibodies (Table 6). Protein bands were detected using SuperSignal West Femto maximum sensitivity substrate (Thermo Fisher Scientific, Rockford, IL, USA) and quantified using Image Lab™ software (version 5.2.1).

TABLE 6

| | | Product | |
|---|---|---|---|
| Group | Antibody Name | Number | Manufacturer |
| Primary | anti-tyrosinase/rabbit | BS6754 | BioWorld |
| Antibody | anti-TRP2/rabbit | BS3320 | |
| | anti-TRP1/rabbit | sc-25543 | Santa Cruz |
| | anti-MITF/mouse | sc-515925 | |
| Secondary | Anti-mouse IgG, HRP-linked | #7076 | Cell Signaling |
| Antibody | Antibody | | |
| | Anti-rabbit IgG, HRP-linked | #7074 | |
| | Antibody | | |

TABLE 7

| | Positive | | | |
|---|---|---|---|---|
| Group | Control | A2 | QC | AQ |
| Concentration | 1 mM | 50 μM | 20 μM | 20 μM:20 μM |

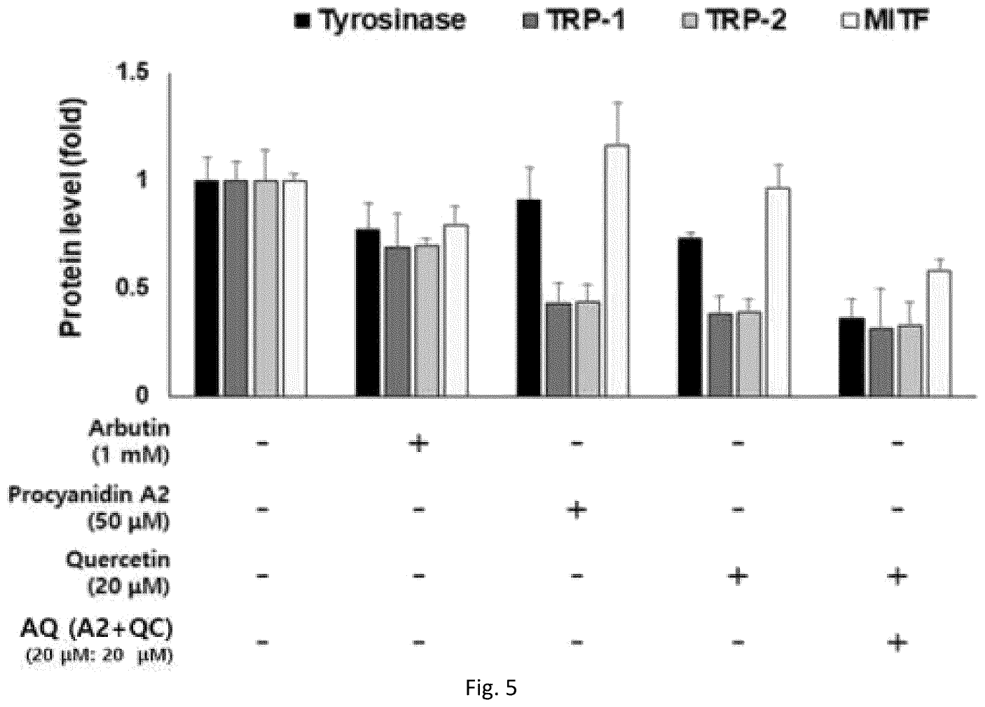

As shown in FIG. 5, the treatment with procyanidin A2 and quercetin separately led to a reduction in the protein expression of tyrosinase, TRP-1, and TRP-2. However, the combination of these compounds resulted in a more significant decrease in protein expression than when each compound was used individually, and even more so compared to the positive control group treated with arbutin. This outcome, which mirrors the results seen in Example 5, demonstrates the superior inhibitory activity of the present invention's combination on melanin synthesis, achieving this effect with a dosage approximately 1/25th of the positive control group.

### Example 6. Analysis of Autophagy Activity-Related Protein Expression Levels Following Treatment with Procyanidin A2, Quercetin, and Their Combination

The mechanism of autophagy, which self-eliminates unnecessary substances, waste, and degenerative proteins within the cell, suppresses melanin production and affects pigment degradation. Consequently, if autophagy is activated by the combination, it could result in preventing, inhibiting, or alleviating pigmentation effects.

LC3-II, ATG5, and Beclin-1 are involved in the formation of autophagosomes and activate autophagy. In contrast, p62 and LC3-I, which show characteristics of being decomposed during autophagosome formation, decrease when autophagy activate. To verify the autophagy activity of procyanidin A2, quercetin, and their combination, the expression levels of autophagy-activity-related proteins were analyzed. The specific method for this experiment, except for using Table 8 for antibodies, was conducted in the same manner as the method in Example 5 for the analysis of melanin synthesis-related protein expression.

TABLE 8

| | | Product | |
|---|---|---|---|
| Group | Antibody Name | Number | Manufacturer |
| Primary | LC3B (D11)/Rabbit mAb | #3868 | Cell Signaling |
| Antibody | Atg5 (D5G3) Rabbit mAb | #9980 | |
| | SQSTM1/p62 (D6M5X) Rabbit | #23214 | |
| | Beclin-1 (D40C5) Rabbit | #3495 | |
| Secondary | Anti-rabbit IgG, HRP-linked | #7074 | |
| Antibody | Antibody | | |

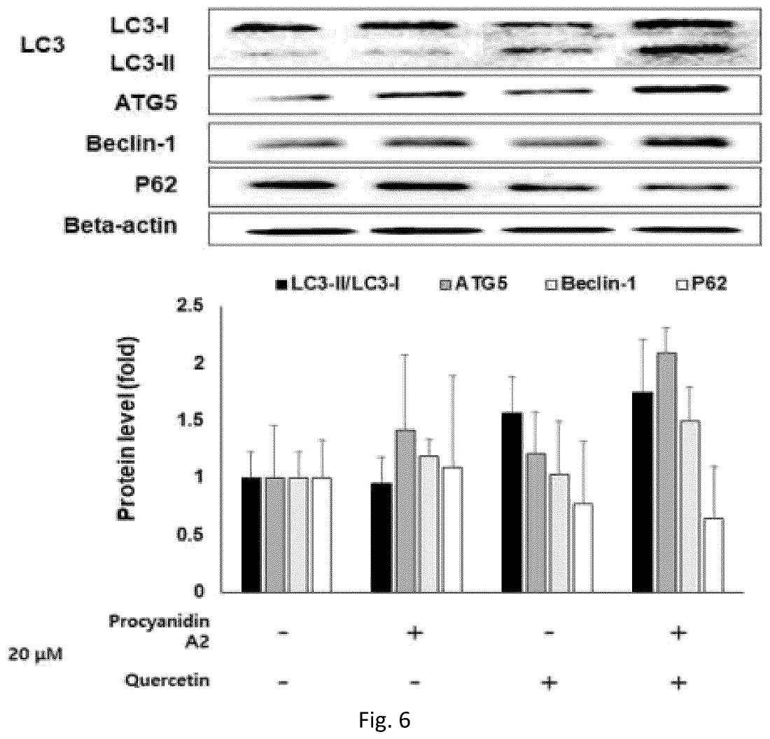

It was observed in FIG. 6 that when procyanidin A2 (50 μM) and quercetin (20 μM) were treated separately, the expression levels of LC3-II, ATG5, and Beclin-1 showed little to no significant change or only slight increases. However, when both compounds (20 μM:20 μM each) were combined, the expression levels of LC3-II, ATG5, and Beclin-1 significantly increased, while those of p62 and LC3-I considerably decreased.

Additionally, treatment with only procyanidin A2 showed a slight increase in P62 which typically decreases with autophagy activation, and a slight increase in ATG5 and Beclin-1 which typically increases with autophagy activation. The ratio of LC3-II to LC3-I, which should respectively increase and decrease, remained similar to the control group. This suggests that procyanidin A2 as a single compound only partially relates to autophagy. However, the combination with quercetin significantly enhanced the influence on autophagy activators, indicating a notably superior synergistic effect of both compounds.

### Example 7. Analysis of Melanin Production Following Treatment with Procyanidin A2, Quercetin, and Their Combination with Autophagy Inhibitors

Wortmannin, bafilomycin, and 3-Methyladenine (3-MA) are known as autophagy inhibitors. Wortmannin and 3-MA inhibit the formation of autophagosomes, thereby suppressing autophagy, while bafilomycin impedes the fusion of formed autophagosomes with lysosomes, inhibiting the formation of autolysosomes. Therefore, the combination of these autophagy inhibitors with the compounds of the present invention was used to verify if melanin production inhibition is achievable through autophagy activation.

Specifically, the experiment involved culturing MNT-1 cells in a 24-well plate at 1×10⁵ cells/well at 37° C., in a 5% CO₂ incubator for 24 hours. Subsequently, the cells were treated with the combination and each of the autophagy inhibitors (Wortmannin, 200 μM; bafilomycin, 300 nM; and 3-MA, 5 mM) either separately or simultaneously, and then incubated for 72 hours. Afterward, the media was removed, cells were dissolved in 1 N NaOH, heated at 95° C. for 5 minutes, and the absorbance was measured at 490 nm using a microplate reader (Molecular Devices, Sunnyvale, CA, USA). The measured absorbance was calculated as a percentage of melanin production, with the amount produced in the non-treatment group (NT) set as 100%.

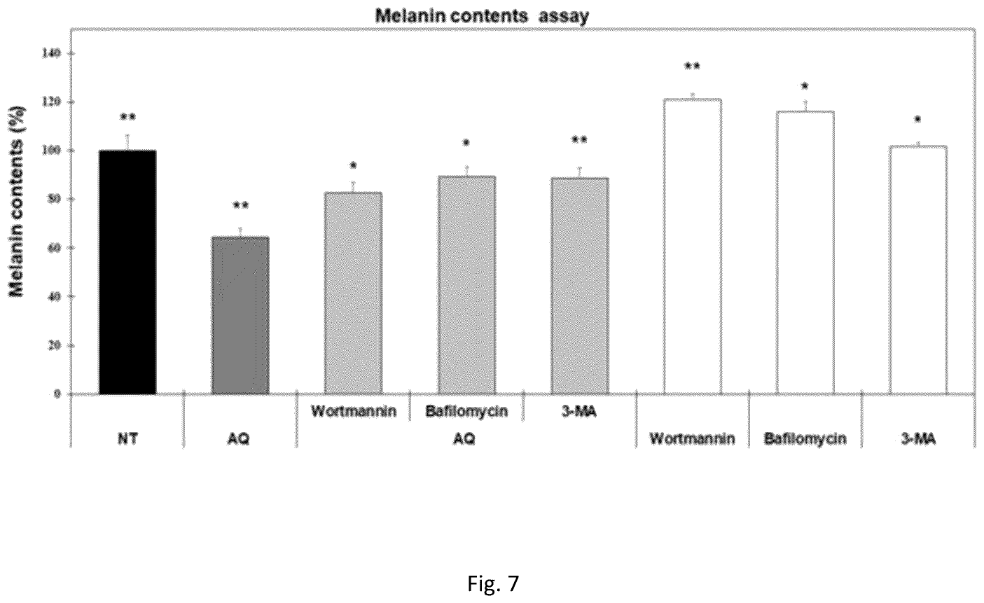

As illustrated in FIG. 7, melanin contents decreased to approximately 60% with the treatment of the combination, whereas it increased to about 80-90% when treated in conjunction with autophagy inhibitors. Moreover, treatment with inhibitors alone resulted in an increase in melanin production compared to the non-treated group.

The description of the present invention as stated above is for illustrative purposes only, and a person having ordinary skill in the art to which the present invention pertains will understand that the technical idea or essential characteristics of the present invention can be easily modified into other specific forms without changing them. Therefore, the examples described above should be understood as illustrative in all aspects and not limited.

1. A method for skin whitening, or treating or alleviating skin pigmentation, comprising: administering a composition comprising procyanidin A2 and quercetin as active ingredients to a subject in need thereof.

2. (canceled)

3. The method of claim 1, wherein the procyanidin A2 and the quercetin are in a weight ratio of 1:2 to 2:1.

4. The method of claim 1, wherein the composition comprises procyanidin A2 and quercetin each at a concentration of 20 μM.

5. The method of claim 1, wherein the procyanidin A2 is in an amount greater than 0 and less than or equal to 50 μM.

6. The method of claim 1, wherein the quercetin is in an amount greater than 0 and less than or equal to 25 μM.

7. The method of claim 1, wherein the composition inhibits the activity of tyrosinase.

8. The method of claim 1, wherein the composition inhibits the amount of melanin produced.

9. The method of claim 1, wherein the composition decreases the expression of one or more melanin synthesis-related genes selected from the group consisting of tyrosinase, TRP-1, TRP-2, and MITF.

10. The method of claim 1, wherein the composition increases the expression of at least one gene of (a), and decreases the expression of at least one gene of (b):
(a) LC3-II, ATG5, and Beclin-1; and
(b) p62, and LC3-I.

11. The method of claim 1, wherein the composition inhibits an autophagy inhibitor.

12. The method of claim 11, wherein the autophagy inhibitor is one or more selected from the group consisting of wortmannin, bafilomycin, and 3-Methyladenine (3-MA).

13. The method of claim 1, wherein the composition is a health functional food composition, a functional food composition, a cosmetic composition, a quasi-drug composition, or a pharmaceutical composition.

14. The method of claim 13, wherein the cosmetic composition is at least one selected from the group consisting of a flexible cosmetic water, nutrition cosmetic water, gel, essence, spray essence, emulsion, cream, lotion, powder, soap, surfactant-containing cleansing, oil, powder foundation, emulsion foundation, wax foundation, cleanser and bath powder, mask pack, massage cream, hand cream, sunscreen, body lotion, and body cleanser.

15. (canceled)

16. The composition of claim 13, wherein the quasi-drug composition is for non-oral administration.

17. (canceled)

18. The method of claim 1, wherein the skin pigmentation disorder is one or more selected from the group consisting of melasma, freckles, lentigo, seborrheic keratosis, mole, café-au-lait spots, nevus of Ota, blue nevus, hyperpigmented spots, drug-induced hyperpigmentation, gravidic chloasma, and post-inflammatory hyperpigmentation due to wounds or dermatitis.
